(19) United States
(12) Patent Application Publication
(10) Pub. No.: US 2008/0021541 A1
(43) Pub. Date: Jan. 24, 2008

(54) LASER PROCESS TO PRODUCE DRUG DELIVERY CHANNEL IN METAL STENTS

(75) Inventor: Richard J. Saunders, Redwood, CA (US)

Correspondence Address: FULWIDER PATTON, LLP (ABBOTT), 6060 CENTER DRIVE, 10TH FLOOR, LOS ANGELES, CA, 90045, US

(73) Assignee: ADVANCED CARDIOVASCULAR SYSTEMS, INC., SANTA CLARA, CA (US)

(21) Appl. No.: 11/831,626

(22) Filed: Jul. 31, 2007

Related U.S. Application Data
(62) Division of application No. 10/939,280, filed on Sep. 10, 2004.

(51) Int. Cl. A61F 2/06

(52) U.S. Cl. 623/1.15; 607/89

(57) ABSTRACT

A method for forming a stent and for also forming channels in the outer surface of selected regions of the stent structure. The method includes impinging a laser beam generated by a diode pumped Q-switched pulsed Nd/YAG laser operating at the third harmonic on an outer surface of a stent and controllably machining channels in the outer surface of the stent. The depth of the channels may be controlled by adjusting the power and pulse rate of the laser, and also by adjusting the rate at which the stent moves relative to the laser beam.

### BACKGROUND OF THE INVENTION

1. Field of the Invention

The present invention relates generally to implantable medical devices and to a method for manufacturing implantable medical devices capable of retaining therapeutic materials and dispensing the therapeutic materials to a desired location of a patient's body. More particularly, the present invention relates to an implantable medical device, such as a stent or other intravascular or intraductal medical device, and to a method for forming channels, depots, holes or other indented structures in the structure of the stent or intravascular or intraductal medical device capable of holding a therapeutic material that is dispensed from the stent or other medical device when the stent or other medical device is implanted within a lumen or duct of the patient.

2. General Background and State of the Art

In a typical percutaneous transluminal coronary angioplasty (PTCA) for compressing lesion plaque against the artery wall to dilate the artery lumen, a guiding catheter is percutaneously introduced into the cardiovascular system of a patient through the brachial or femoral arteries and advanced through the vasculature until the distal end is in the ostium. A dilatation catheter having a balloon on the distal end is introduced through the catheter. The catheter is first advanced into the patient's coronary vasculature until the dilatation balloon is properly positioned across the lesion.

Once in position across the lesion, a flexible, expandable, preformed balloon is inflated to a predetermined size at relatively high pressures to radially compress the atherosclerotic plaque of the lesion against the inside of the artery wall and thereby dilate the lumen of the artery. The balloon is then deflated to a small profile, so that the dilatation catheter can be withdrawn from the patient's vasculature and blood flow resumed through the dilated artery. While this procedure is typical, it is not the only method used in angioplasty.

In angioplasty procedures of the kind referenced above, restenosis of the artery often develops which may require another angioplasty procedure, a surgical bypass operation, or some method of repairing or strengthening the area. To reduce the likelihood of the development of restenosis and strengthen the area, a physician can implant an intravascular prosthesis, typically called a stent, for maintaining vascular patency. In general, stents are small, cylindrical devices whose structure serves to create or maintain an unobstructed opening within a lumen. The stents are typically made of, for example, stainless steel, nitinol, or other materials and are delivered to the target site via a balloon catheter. Although the stents are effective in opening the stenotic lumen, the foreign material and structure of the stents themselves may exacerbate the occurrence of restenosis or thrombosis.

A variety of devices are known in the art for use as stents, including expandable tubular members, in a variety of patterns, that are able to be crimped onto a balloon catheter, and expanded after being positioned intraluminally on the balloon catheter, and that retain their expanded form. Typically, the stent is loaded and crimped onto the balloon portion of the catheter, and advanced to a location inside the artery at the lesion. The stent is then expanded to a larger diameter, by the balloon portion of the catheter, to implant the stent in the artery at the lesion. Typical stents and stent delivery systems are more fully disclosed in U.S. Pat. No. 5,514,154 (Lau et al.), U.S. Pat. No. 5,507,768 (Lau et al.), and U.S. Pat. No. 5,569,295 (Lam et al.).

Stents are commonly designed for long-term implantation within the body lumen. Some stents are designed for non-permanent implantation within the body lumen. By way of example, several stent devices and methods can be found in commonly assigned and common owned U.S. Pat. No. 5,002,560 (Machold et al.), U.S. Pat. No. 5,180,368 (Garrison), and U.S. Pat. No. 5,263,963 (Garrison et al.).

Intravascular or intraductal implantation of a stent generally involves advancing the stent on a balloon catheter or a similar device to the designated vessel/duct site, properly positioning the stent at the vessel/duct site, and deploying the stent by inflating the balloon which then expands the stent radially against the wall of the vessel/duct. Proper positioning of the stent requires precise placement of the stent at the vessel/duct site to be treated. Visualizing the position and expansion of the stent within a vessel/duct area is usually done using a fluoroscopic or x-ray imaging system.

Although PTCA and related procedures aid in alleviating intraluminal constrictions, such constrictions or blockages reoccur in many cases. The cause of these recurring obstructions, termed restenosis, is due to the body's immune system responding to the trauma of the surgical procedure. As a result, the PTCA procedure may need to be repeated to repair the damaged lumen.

In addition to providing physical support to passageways, stents are also used to carry therapeutic substances for local delivery of the substances to the damaged vasculature. For example, anticoagulants, antiplatelets, and cytostatic agents are substances commonly delivered from stents and are used to prevent thrombosis of the coronary lumen, to inhibit development of restenosis, and to reduce post-angioplasty proliferation of the vascular tissue, respectively. The therapeutic substances are typically either impregnated into the stent or carried in a polymer that coats the stent. The therapeutic substances are released from the stent or polymer once it has been implanted in the vessel.

Drugs or similar agents that limit or dissolve plaque and clots are used to reduce, or in some cases eliminate, the incidence of restenosis and thrombosis. The term “drug(s),” as used herein, refers to all therapeutic agents, diagnostic agents/reagents and other similar chemical/biological agents, including combinations thereof, used to treat and/or diagnose restenosis, thrombosis and related conditions. Examples of various drugs or agents commonly used include heparin, hirudin, antithrombogenic agents, steroids, ibuprofen, antimicrobials, antibiotics, tissue plasma activators, monoclonal antibodies, and antifibrosis agents.

Since the drugs are applied systemically to the patient, they are absorbed not only by the tissues at the target site, but by all areas of the body. As such, one drawback associated with the systemic application of drugs is that areas of the body not needing treatment are also affected. To provide more site-specific treatment, stents are frequently used as a means of delivering the drugs exclusively to the target site. The drugs are suspended in a tissue-compatible polymer, such as silicone, polyurethane, polyvinyl alcohol, polyethylene, polyesters, hydrogels, hyaluronate, various copolymers and blended mixtures thereof. The polymer matrix is applied to the surfaces of the stent generally during the manufacture of the stent. By positioning the stent at the target site, the drugs can be applied directly to the area of the lumen requiring therapy or diagnosis.

In addition to the benefit of site-specific treatment, drug-loaded stents also offer long-term treatment and/or diagnostic capabilities. These stents include a biodegradable or absorbable polymer suspension that is saturated with a particular drug. In use, the stent is positioned at the target site and retained at that location either for a predefined period or permanently. The polymer suspension releases the drug into the surrounding tissue at a controlled rate based upon the chemical and/or biological composition of the polymer and drug.

A problem with delivering therapeutic substances from a stent is that, because of the limited size of the stent, the total amount of therapeutic substance that can be carried by the stent is limited. Furthermore, when the stent is implanted into a blood vessel, much of the released therapeutic substance enters the blood stream before it can benefit the damaged tissue. To improve the effectiveness of the therapeutic substances, it is desirable to maximize the amount of therapeutic substance that enters the local vascular tissue and minimize the amount that is swept away in the bloodstream.

What has been needed, and heretofore unavailable, is an efficient and cost-effective method of forming reservoirs in the structure of a stent for holding larger volumes of therapeutic substances than are possible where the stent is simply coated with the substance. The present invention satisfies this, and other needs.

### SUMMARY OF THE INVENTION

Briefly, and in general terms, the present invention provides a method and apparatus for machining the outer surface of a stent structure using a laser. More specifically, a laser, such as, for example, but not limited to, a diode pumped Q-switched laser emitting light at a third harmonic, is used to selectively and controllably machine a channel into the outer surface of a stent. The width of the channel may be controlled by varying the spot size of the laser beam, and the depth of the channel is controlled by controlling the spot size of the beam, the power of the beam, the pulse frequency, and the rate of relative motion between the beam and the stent. The channels may be filled with a therapeutic substance, thus acting as a reservoir for delivering the therapeutic substance to the wall of a vessel of a person.

In another aspect, the present invention provides a system and method wherein the laser and stent move relative to each other using computer controlled CNC X/Y precision equipment as is know to those skilled in the art. In one aspect, a Nd/YAG laser may be used to cut a stent pattern into a tubular member of a suitable material, and the diode pumped Q-switched laser is used to machine the channels into the structure of the stent before the stent pattern has been cut out.

In yet another aspect of the present invention, the Nd/YAG and diode pumped Q-switched lasers are mounted on the same cutting apparatus such that the laser beams utilize the same positioning system. In this manner, registration inaccuracies associated with removal of the stent from the stent pattern cutting equipment and remounting the stent in the channel machining equipment are avoided.

In another aspect, one laser, such as, for example, a diode pumped Q-switched laser emitting light at a third harmonic, may be used to machine both the channels and the structure of the stent.

In still another aspect of the present invention, a channel having a selected depth may be machined into a stent structure in a single pass under the laser beam. In an alternative aspect, the depth of channel may be selectively deepened by moving the stent structure under the laser beam for one or more additional passes. Thus the capacity of the channels, and hence the amount of therapeutic substance that the channel may contain, may be varied as desired to provide more or less therapeutic substance for delivery to the wall of a body vessel. In yet another aspect, the channels may be machined with either continuously varying depths, or depths that vary in discrete amounts at selected locations on the structure of the stent.

In a still further aspect of the present invention, the method includes delaying exposing the stent structure to the channel cutting laser beam for a selected period of time after beginning to move the stent relative to the laser beam. This method is advantageous in that it accommodates the lag in motion of the precision machinery relative to the initiation of the laser beam that may result in the beginning portion of the channel having greater depth than a portion of the channel that was exposed to the laser beam after the relative motion between the stent and the laser beam has begun.

These and other advantages and features of the present invention will be apparent from the following detailed description when taken in conjunction with the accompanying drawings of exemplary embodiments.

### BRIEF DESCRIPTION OF THE DRAWINGS

FIG. 1 is an elevational view, partially in section, of a stent embodying features of the invention which is mounted on a delivery catheter and disposed within a damaged artery.

FIG. 2 is an elevational view, partially in section, similar to that shown in FIG. 1 wherein the stent is expanded within a damaged artery, pressing the damaged lining against the arterial wall.

FIG. 3 is an elevational view, partially in section showing the expanded stent within the artery after withdrawal of the delivery catheter.

FIG. 4 is a perspective view of a stent embodying in an unexpanded state, with one end of the stent being shown in an exploded view to illustrate the details thereof.

FIG. 5 is a plan view of a flattened section of a stent of the invention which illustrates the undulating pattern of the stent shown in FIG. 4.

FIG. 5a is a sectional view taken along the line 5a-5a in FIG. 5.

FIG. 6 is a schematic representation of equipment for selectively cutting the tubing in the manufacture of stents, in accordance with the present invention.

FIG. 7 is an elevational view of a system for cutting an appropriate pattern by laser in a metal tube to form a stent and to machine channels into the structure of the stent in accordance with the invention.

FIG. 8 is a plan view of the laser head and optical delivery subsystem for the laser cutting system shown in FIG. 7.

FIG. 9 is an elevational view of a coaxial gas jet, rotary collet, tube support and beam blocking apparatus for use in the system of FIG. 7.

FIG. 10 is a sectional view taken along the line 10-10 in FIG. 9.

FIG. 11 is a schematic diagram of a diode pumped Q-Switched Nd/YAG laser configured to emit light in the UV region at the third harmonic.

FIG. 12 is an enlarged overhead view of a portion of a stent incorporating channels machined into the outer surface of the stent in accordance with the embodiments of the present invention.

FIG. 13 is a cross-sectional view taken along line 13-13 of FIG. 12 illustrating a profile of a channel formed in accordance with an embodiment of the present invention.

FIG. 14 is a cross-sectional side view taken along line 14-14 of FIG. 12 illustrating a profile of a channel formed in accordance with one embodiment of the present invention.

### DETAILED DESCRIPTION OF THE PREFERRED EMBODIMENTS

To assist in understanding the present invention, it is useful to first describe a typical stent, the manner in which it is mounted on a catheter for implantation in a vessel lumen, and a procedure typically used for carrying out the implantation. While one particular stent design is used for illustration, those skilled in the art will understand that the structure and method of the present invention may be applied to any stent design capable of having reservoirs, which may be filled with a therapeutic substance, formed in an outer surface of the stent.

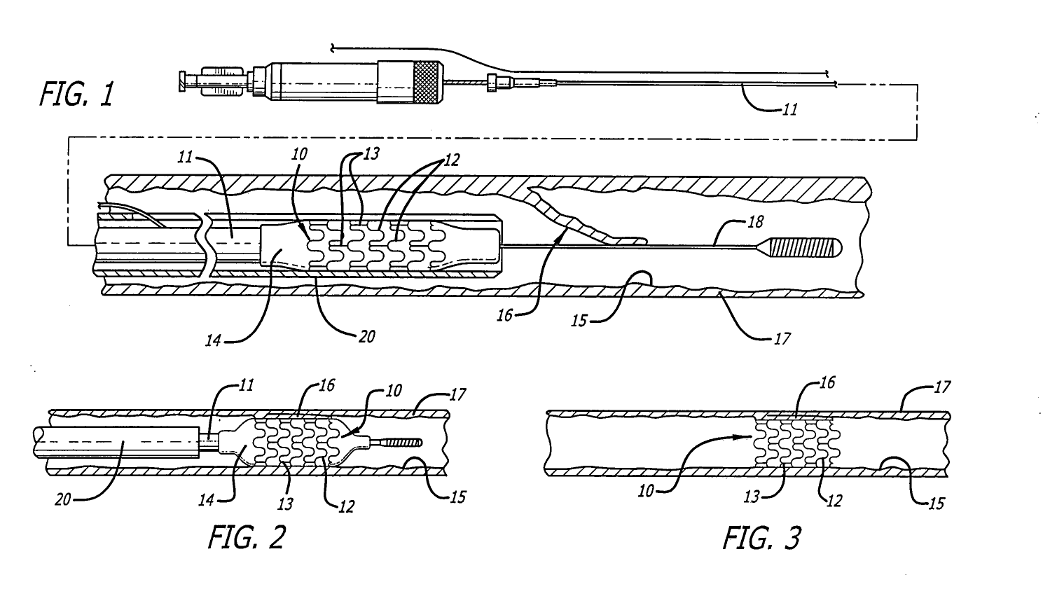

Referring now to the drawings, and particularly FIG. 1 thereof, there is shown a stent 10 which is mounted onto a delivery catheter 11. The stent 10 is a high precision patterned tubular device. The stent 10 typically comprises a plurality of radially expanded cylindrical elements 12 disposed generally coaxially and interconnected by elements 13 disposed between adjacent cylindrical elements. The delivery catheter 11 has an expandable portion or balloon 14 for expanding of the stent 10 within an artery 15. The artery 15, as shown in FIG. 1 has a dissected lining 16 which has occluded a portion of the arterial passageway.

The typical delivery catheter 11 onto which the stent 10 is mounted, is essentially the same as a conventional balloon dilatation catheter for angioplasty procedures. The balloon 14 may be formed of suitable materials such as polyethylene, polyethylene terephthalate, polyvinyl chloride, nylon and ionomers such as Surlyn®, manufactured by the Polymer Products Division of the Du Pont Company. Other polymers may also be used. In order for the stent 10 to remain in place on the balloon 14 during delivery to the site of the damage within the artery 15, the stent 10 is compressed onto the balloon. In one embodiment, a retractable protective delivery sleeve 20 may be provided to further ensure that the stent stays in place on the expandable portion of the delivery catheter 11 and prevent abrasion of the body lumen by the open surface of the stent 20 during delivery to the desired arterial location. Other means for securing the stent 10 onto the balloon 14 may also be used, such as providing collars or ridges on the ends of the working portion, i.e. the cylindrical portion, of the balloon.

Each radially expandable cylindrical element 12 of the stent 10 may be independently expanded. Therefore, the balloon 14 may be provided with an inflated shape other than cylindrical, e.g. tapered, to facilitate implantation of the stent 10 in a variety of body lumen shapes.

The delivery of the stent 10 is accomplished in the following manner. The stent 10 is first mounted onto the inflatable balloon 14 on the distal extremity of the delivery catheter 11. The balloon 14 is slightly inflated to secure the stent 10 onto the exterior of the balloon. The catheter-stent assembly is introduced within the patient's vasculature in a conventional Seldinger technique through a guiding catheter (not shown). A guidewire 18 is disposed across the damaged arterial section with the detached or dissected lining 16 and then the catheter-stent assembly is advanced over a guidewire 18 within the artery 15 until the stent 10 is directly under the detached lining 16. The balloon 14 of the catheter is expanded, expanding the stent 10 against the artery 15, which is illustrated in FIG. 2. While not shown in the drawing, the artery 15 is preferably expanded slightly by the expansion of the stent 10 to seat or otherwise fix the stent 10 to prevent movement. In some circumstances during the treatment of stenotic portions of an artery, the artery may have to be expanded considerably in order to facilitate passage of blood or other fluid therethrough.

The stent 10 serves to hold open the artery 15 after the catheter 11 is withdrawn, as illustrated by FIG. 3. Due to the formation of the stent 10 from elongated tubular member, the undulating component of the cylindrical elements of the stent 10 is relatively flat in transverse cross-section, so that when the stent is expanded, the cylindrical elements are pressed into the wall of the artery 15 and as a result do not interfere with the blood flow through the artery 15. The cylindrical elements 12 of the stent 10 which are pressed into the wall of the artery 15 will eventually be covered with endothelial cell growth which further minimizes blood flow interference. The undulating portion of the cylindrical sections 12 provide good tacking characteristics to prevent stent movement within the artery. Furthermore, the closely spaced cylindrical elements 12 at regular intervals provide uniform support for the wall of the artery 15, and consequently are well adapted to tack up and hold in place small flaps or dissections in the wall of the artery 15, as illustrated in FIGS. 2 and 3.

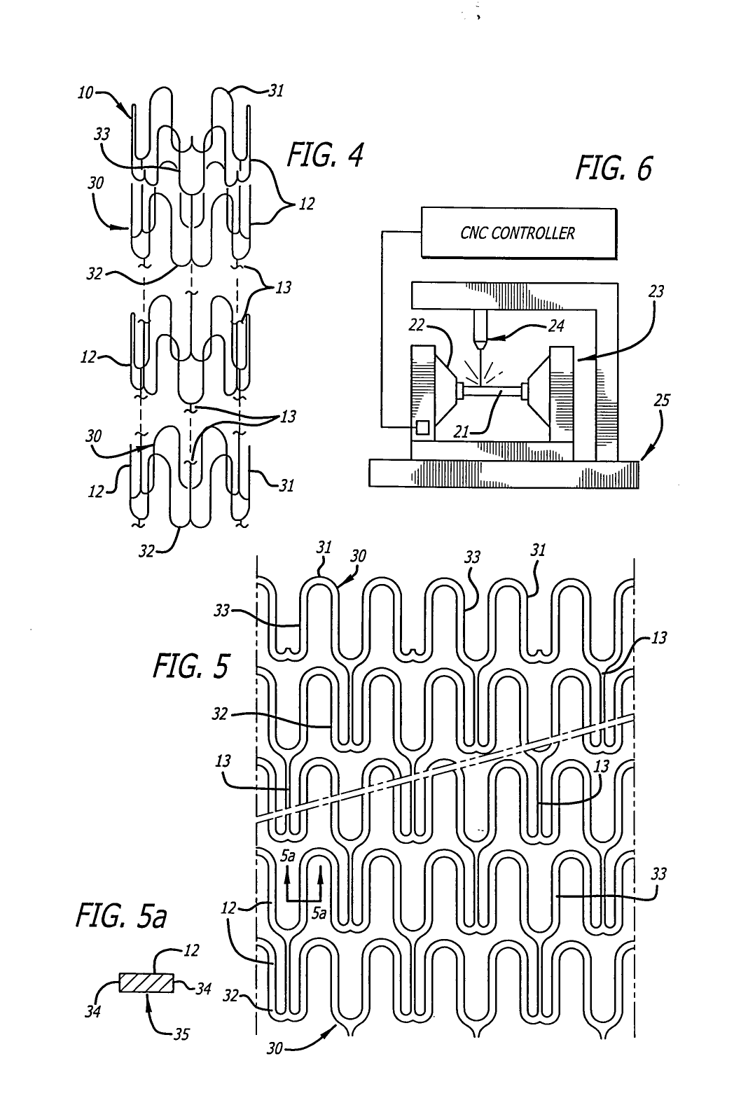

FIG. 4 is an enlarged perspective view of the stent 10 shown in FIG. 1 with one end of the stent shown in an exploded view to illustrate in greater detail the placement of interconnecting elements 13 between adjacent radially expandable cylindrical elements 12. Each pair of the interconnecting elements 13 on one side of a cylindrical element 12 are preferably placed to achieve maximum flexibility for a stent. In the embodiment shown in FIG. 4, the stent 10 has three interconnecting elements 13 between adjacent radially expandable cylindrical elements 12 which are 120 degrees apart. Each pair of interconnecting elements 13 on one side of a cylindrical element 12 are offset radially 60 degrees from the pair on the other side of the cylindrical element. The alternation of the interconnecting elements results in a stent which is longitudinally flexible in essentially all directions. Various configurations for the placement of interconnecting elements are possible. Typically, all of the interconnecting elements of an individual stent are secured to either the peaks or valleys of the undulating structural elements in order to prevent shortening of the stent during the expansion thereof.

The number of undulations may also be varied to accommodate placement of interconnecting elements 13, e.g. at the peaks of the undulations or along the sides of the undulations as shown in FIG. 5.

As best observed in FIGS. 4 and 5, cylindrical elements 12 are in the form of a serpentine pattern 30. As previously mentioned, each cylindrical element 12 is connected by interconnecting elements 13. Serpentine pattern 30 is made up of a plurality of U-shaped members 31, W-shaped members 32, and Y-shaped members 33, each having a different radius so that expansion forces are more evenly distributed over the various members.

The illustrative stent 10 and similar stent structures can be made in many ways. For example, one preferred method of making the stent is to cut a thin-walled tubular member, such as stainless steel tubing to remove portions of the tubing in the desired pattern for the stent, leaving relatively untouched the portions of the metallic tubing which are to form the stent. Generally, the tubing is cut in the desired pattern by means of a machine-controlled laser as illustrated schematically in FIG. 6.

The tubing may be made of suitable biocompatible material such as stainless steel. The stainless steel tube may be Alloy type: 316L SS, Special Chemistry per ASTM F138-92 or ASTM F139-92 grade 2. Special Chemistry of type 316L per ASTM F138-92 or ASTM F139-92 Stainless Steel for Surgical Implants in weight percent. Alternatively, the tubing may be made a material such as cobalt chromium.

The stent diameter is very small, so the tubing from which it is made must necessarily also have a small diameter. Typically the stent has an outer diameter on the order of about 0.075 inch in the unexpanded condition, the same outer diameter of the tubing from which it is made, and can be expanded to an outer diameter of 0.1 inch or more. The wall thickness of the tubing is in the range of about 0.003 inch to 0.007 inch, and preferably in the range of 0.003 inch to 0.005 inch.

Referring to FIG. 6, the tubing 21 is put in a rotatable collet fixture 22 of a machine-controlled apparatus 23 for positioning the tubing 21 relative to a laser 24. According to machine-encoded instructions, the tubing 21 is rotated and moved longitudinally relative to the laser 24 which is also machine controlled. The laser selectively removes the material from the tubing by ablation and a pattern is cut into the tube. The tube is therefore cut by the laser into the discrete pattern of the finished stent.

The process of cutting a pattern for the stent into the tubing is automated except for loading and unloading the length of tubing. Referring again to FIG. 6 it may be done, for example, using a CNC-opposing collet fixture 22 for axial rotation of the length of tubing, in conjunction with a CNC X/Y table 25 to move the length of tubing axially relatively to a machine-controlled laser as described. The entire space between collets can be patterned using the CO₂ laser set-up of the foregoing example. The program for control of the apparatus is dependent on the particular configuration used and the pattern to be ablated in the coating.

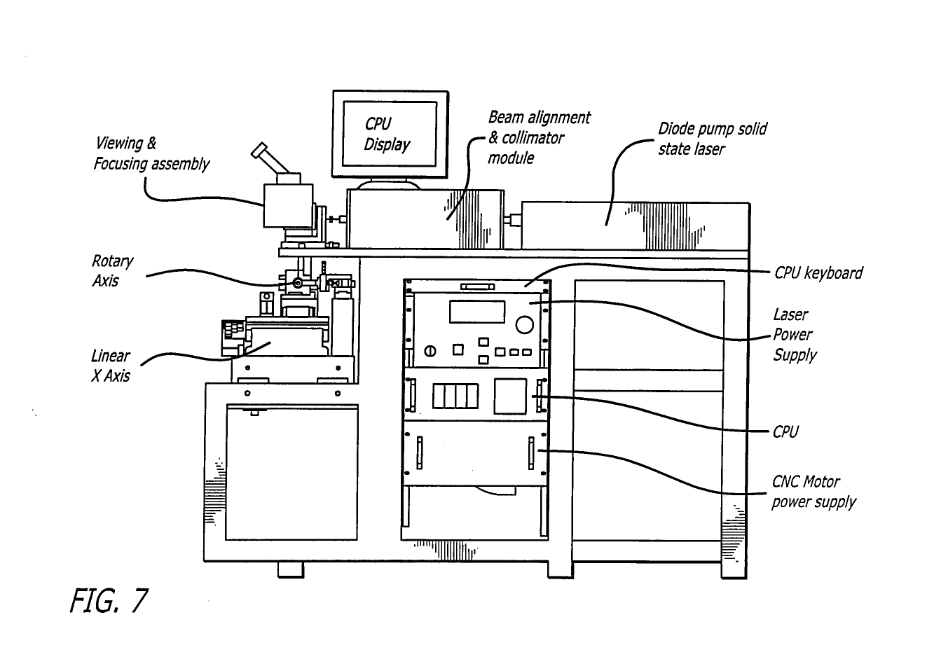

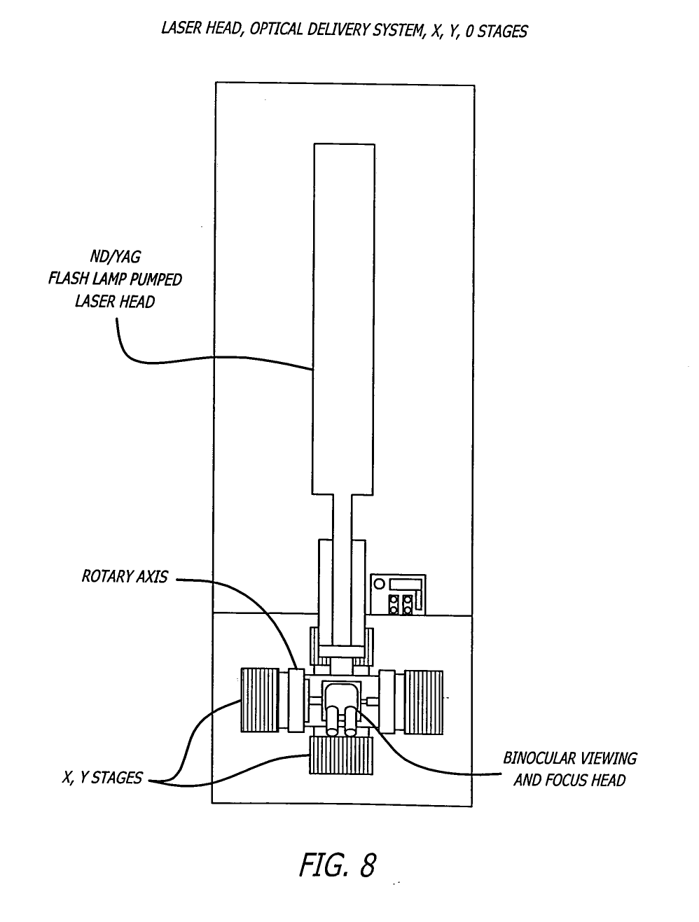

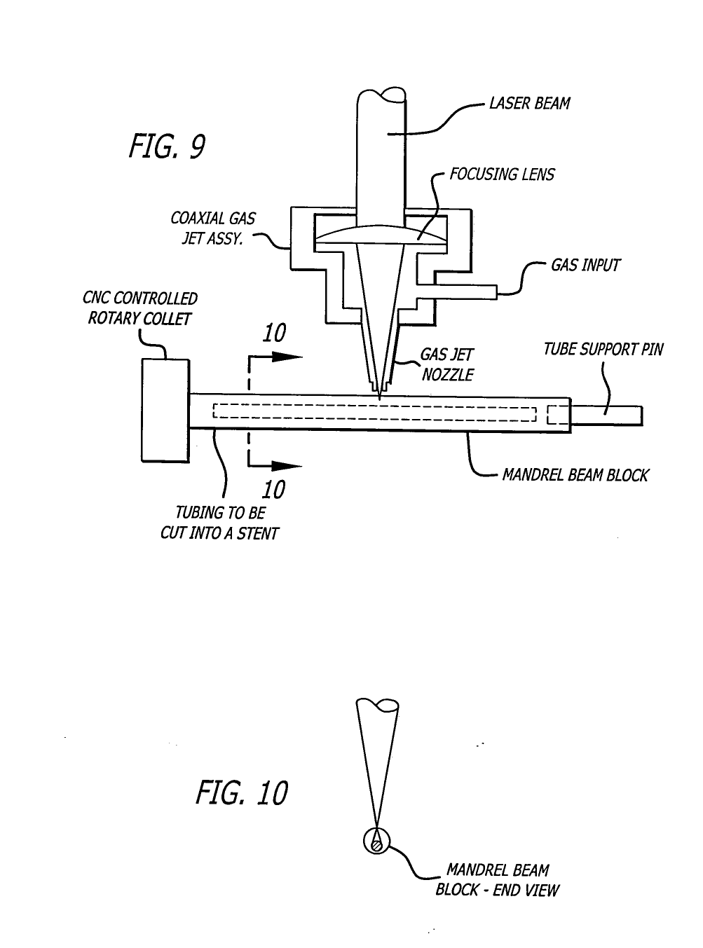

Referring now to FIGS. 7-10 of the drawings, there is illustrated a process and apparatus for producing metal stents with a fine precision structure cut from a small diameter thin-walled cylindrical tube. Cutting a fine structure, such as, for example, on the order of approximately 0.0035″ web width, or less, requires minimal heat input and the ability to manipulate the tube with precision. It is also necessary to support the tube yet not allow the stent structure to distort during the cutting operation.

The tube from which the stent is cut is typically made of stainless steel or cobalt chromium with an outside diameter of, for example, 0.060″ to 0.080″ and a wall thickness of, for example, 0.002″ to 0.007″. These tubes are fixtured under a laser and positioned utilizing a CNC to generate a very intricate and precise pattern. Due to the thin wall thickness and the small geometry of the stent pattern, it is necessary to have very precise control of the laser, its power level, the focused spot size, and the precise positioning of the laser cutting path to ensure that the geometry of the structure left behind after the laser cuts out the stent pattern is acceptable and not distorted or damaged in such a manner as to affect the integrity of the finished stent.

In order to minimize the heat input into the stent structure, and thus minimize thermal distortion of the tube, uncontrolled burn out of the metal, and metallurgical damage due to excessive heat, and thereby produce a smooth debris free cut, a Nd/YAG laser, such as, for example, a Nd/YAG laser available from LASAG, Arlington Heights, Ill., produces short pulses in the range of 0.075 milliseconds to 0.150 milliseconds, and preferably in the range of 0.05 to 0.150 milliseconds. The pulse frequency is typically in the range of 2 kHz, and the power of the laser may be adjusted to provide optimum cutting/machining of the desired fine structures and channels. With this laser and pulse widths, it is possible to make smooth, narrow cuts in the stainless of cobalt chromium tubes in very fine geometries without damaging the narrow struts that make up the stent structure. Such a system makes it possible to adjust the laser parameters to cut narrow a kerf width which will minimize the heat input into the material.

The positioning of the tubular structure requires the use of precision CNC equipment such as, for example, that manufactured and sold by Aerotech, Inc. of Pittsburgh, Pa. In addition, a rotary mechanism is provided that allows the computer program to be written as if the pattern were being cut from a flat sheet. This allows both circular and linear interpolation to be utilized in programming. Since the finished structure of the stent is very small, a precision drive mechanism is required. The optical system which expands the original laser beam, delivers the beam through a viewing head and focuses the beam onto the surface of the tube, incorporates a coaxial gas jet and nozzle that helps to remove debris from the kerf and cools the region where the beam interacts with the material as the beam cuts and vaporizes the metal. It is also necessary to block the beam as it cuts through the top surface of the tube and prevent the beam, along with the molten metal and debris from the cut, from impinging on the opposite surface of the tube.

In addition to the laser and the CNC positioning equipment, the optical delivery system includes a beam expander to increase the laser beam diameter, a binocular viewing head and focusing lens, and a coaxial gas jet that provides for the introduction of a gas stream that surrounds the focused beam and is directed along the beam axis. The delivery system may also include a circular polarizer, typically in the form of a quarter wave plate, to eliminate polarization effects in metal cutting.

The coaxial gas jet nozzle, typically having a small inner diameter, for example, 0.018″ I.D., is centered around the focused beam with approximately 0.025″ between the tip of the nozzle and the tubing. The jet is pressurized with a gas, such as, for example, air or oxygen at, for example, 20 psi and is directed at the tube with the focused laser beam exiting the tip of the nozzle. The gas reacts with the metal to assist in the cutting process. In this manner, it is possible to cut the material with a very fine kerf with precision. In order to prevent burning by the beam and/or molten slag on the far wall of the tube I.D., a stainless steel mandrel, having, for example, a diameter of approximately 0.034 inches may be placed inside the tube and allowed to roll on the bottom of the tube as the pattern is cut. This acts as a beam/debris block protecting the far wall I.D. Protection of the far wall I.D. may also be accomplished by inserting a second tube inside the stent tube which has an opening to trap the excess energy in the beam which is transmitted through the kerf along which collecting the debris that is ejected from the laser cut kerf. A vacuum or positive pressure can be placed in this shielding tube to remove the collection of debris. The laser cutting process results in a very narrow kerf, on the order of approximately 0.001 inches.

In most cases, the gas utilized in the jets may be reactive or non-reactive (inert). In the case of reactive gas, oxygen or compressed air is used. For example, compressed air may be used since it offers more control of the material removed and reduces the thermal effects of the material itself. Inert gas such as argon, helium, or nitrogen can also be used to eliminate any oxidation of the cut material. The result is a cut edge with no oxidation, but there is usually a tail of molten material that collects along the exit side of the gas jet that must be mechanically or chemically removed after the cutting operation.

Generally, the cut stent is electrochemically polished in an acidic aqueous solution after laser cutting. For example, stents cut from stainless steel tubing are electropolished in a solution such as ELECTRO-GLO#300, sold by the ELECTRO-GLO Co., Inc. of Chicago, Ill.

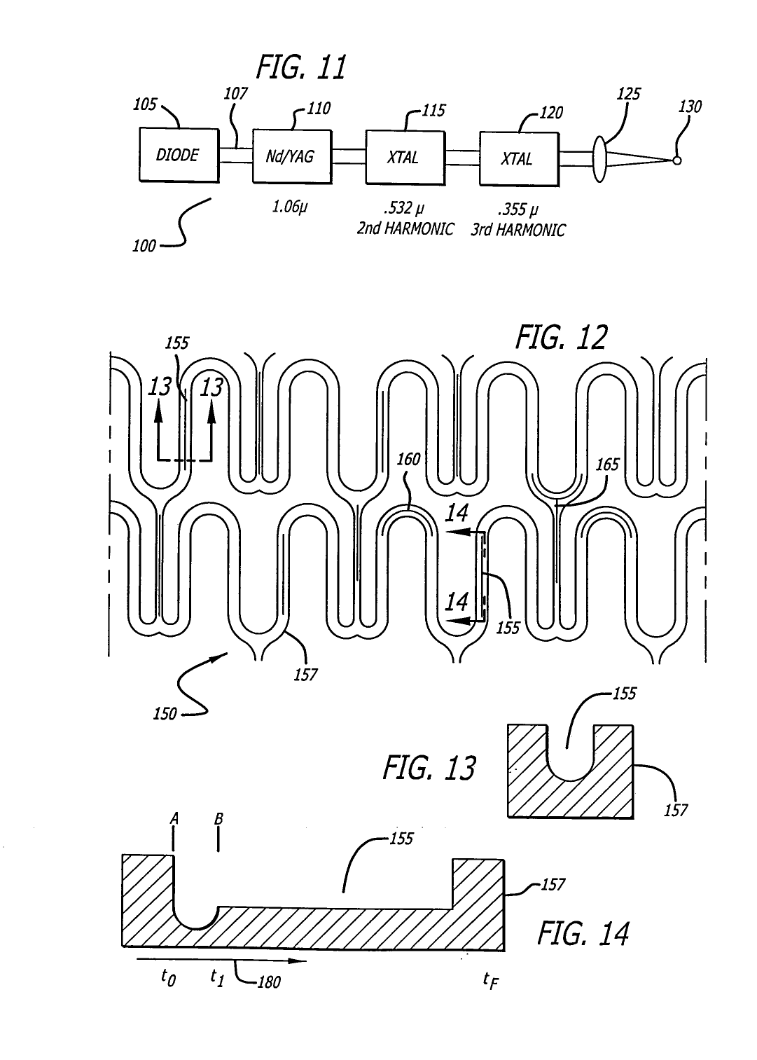

Referring now to FIG. 11, an improved laser system 100 incorporating aspects of the present invention is illustrated for machining depots or channels into the outer surface of the stent structure to form reservoirs to carry increased amounts of the therapeutic substances, and to allow for some control of their release into the wall of the patient's vessel. Laser system 100 comprises a diode laser, such as the AVIA diode pumped Nd/YAG laser manufactured by COHERENT, Inc. This laser is a Q-switched laser having a pulse length in the range of 12 to 40 nanoseconds at frequencies from 1 Hz to 100 kHz. The energy per pulse can be varied from 0.1 to about 250 microjoules, depending on the pulse frequency and diode pump power level.

Light 107 from diode 105 is used to pump the Nd/YAG crystal 110 which them emits light having a wavelength of 1060 nanometers. This light is then transmitted through a frequency doubler crystal 115 and then through conversion crystal 120. Light emitting from crystal 120 is emitted as the third harmonic of the original laser light and has a wavelength of 355 nanometers. Such a light beam is capable of being finely focused using lens or lens system 125 to narrow beam diameter 130, so as to produce a channel in the outer surface of a stent's structure approximately in the range of 20-60 microns in width using a focal distance of approximately 50 to 100 millimeters.

Using the methods of the present invention, a narrow channel of controlled depth can be produced by controlling the position of the beam, the spot size of the laser beam, the frequency of the laser, and the power level of the laser. Typically, the channels or depots are machined into the tubing blank as described above, and then the stent pattern is cut. Alternatively, the stent pattern may be cut first, and then the channels or depots machined into the structure of the stent.

The stainless steel or chromium cobalt tubing is mounted into a rotatable collet fixture of a machine-controlled apparatus positioning the stent relative to the laser. According to machine-encoded instructions, the tubing is rotated and moved longitudinally relative to the laser, which is also machine controlled. During this process, the laser selectively removes material from the outer surface of the tubing, forming channels having a controlled width and depth at selected locations on the outer surface of the tubing.

A gas jet may be used to ensure removal of material from the vicinity of the stent surface. For example, in one embodiment, compressed air at approximately 30 psi can be blown across the stent, or supplied through a coaxial gas jet assembly (FIG. 6.). Use of such a gas jet has been found to reduce the formation of ripples on the bottom surface of the channel, resulting in a smoother bottom surface of the channel.

FIG. 12 illustrates the formation of channels into the outer surface of various portions of the structure 157 of a stent 150. As is apparent, channels may be formed that have many different geometries, such as channel 155 which is machined into a relatively straight portion the stent structure, channel 160 which is machined into a relatively serpentine or rounded portion of the stent structure, and channel 165 where the channel has been machined to have a “Y” configuration, following a similarly shaped structure of the stent. The channels may be continuous, or they may be machined at discreet locations, resulting in a stent structure having channel portions and non-channel portions.

FIG. 13 illustrates the approximate shape of channel 155 taken along line 13 of FIG. 12. Channel 155 is approximately rectangular in cross-section, although the overall shape may vary somewhat depending on the parameters used to carry out the laser machining operation. Additionally, those skilled in the art will understand that the overall cross-sectional shape is modified during electrochemical polishing of the stent.

The depth of the channel may be controlled by controlling the power and pulse frequency of the laser and the speed of the positioning system. For example, in one test, a series of passes along a stent strut were performed, and the depth of the channel was determined after each pass using a profilometer manufactured by VEECO, Inc. The laser was operated at a diode current of 50%, pulse frequency of 1.0 kHz, energy per pulse of 143 microjoules, and an average power of 0.14 watts for each pass. Three passes were made using a feed rate at each of 4 and 6 inches per minute. Compressed air at approximately 30 psi was supplied through a coaxial gas jet assembly, and a lens having a focal length of 75 millimeters was used to focus the beam.

For all tests, the width of the channel was determined to be approximately 40 micrometers after light polishing to clean debris from the channel. The depth of the channel varied depending on the number of passes that were made, and the feed rate, as illustrated below:

| Feed Rate | Pass 1 | Pass 2 | Pass 3 |
|---|---|---|---|
| 4 inches/minute | 8.18 micrometers | 20.37 micrometers | 32.90 |
| | | | micrometers |
| 6 inches/minute | 5.27 micrometers | 12.94 micrometers | 19.38 |
| | | | micrometers |

It will be apparent from the above described example that the laser system and method of the present invention may be operated so as to selectively cut channels of differing depths into the outer surface of the structure of a stent. Thus, the operation of the laser in this fashion is different from the cutting operation used to cut the pattern of the stent out of the tubing. The laser machining process of the present invention provides for much more control over the removal of material from the surface of the stent, and does not result in the production of large amounts of slag or debris that must then be removed from the stent.

Using the methods and system of the present invention, channels having one depth may be cut into a particular area of the stent, such as in the center of the stent, (as located along the longitudinal dimension of the stent), while deeper channels may be machined into the outer structure of the stent at one or both ends of the stent. The depth of the channel may be varied along the length of the channel by varying the power, pulse frequency, and positioning system speed. For example, the channel may be machined to a deeper depth in a selected portion of the stent structure, such as along a straight portion of the stent structure, and machined to a shallower depth in a curved portion of the stent structure, where more strength resulting from a thicker cross-section is need to combat the concentration of stresses that typically occur along the curved portion of the stent structure.

The capability of machining channels having variable depth is also advantageous in that it is thus possible to provide reservoirs holding different amounts of therapeutic substances located in different areas or portions of the stent. Such differential or variable loading of therapeutic substances may be useful in controlling the delivery of the substance to the wall of the patient's vessel. For example, it may be desirable to provide an increased amount of therapeutic substance at the ends of the stent to assist in suppressing restenosis in the area of the vessel wall adjacent to the end of the stent.

In an alternative setup, illustrated in FIG. 15, the Nd/YAG laser emitting 355 nanometer UV light may be used to machine the channels and cut out the stent pattern. This arrangement is particularly advantageous in that if prevents any errors induced in the location of the channels relative to the structure of the stent caused by the necessity of re-registering, or aligning, the stent when it is placed into a separate cutting apparatus.

Using this arrangement, the channels are machined into the stent tubing, and then the stent pattern is cut into the stent as discussed above. Alternatively, the stent pattern may be cut, and then the channels machined along the stent pattern. The machine controlled CNC X/Y table, in accordance with machine-encoded instructions, positions the tubing relative to the laser to controllably machine channels having a selected width and depth into the outer surface of the tubing. Similarly, the positioning system, in accordance with machine-encoded instructions, positions the tubing having the channels machined into it relative to the laser to controllably cut a stent pattern into the stent.

Referring now to FIG. 14, another aspect of the present invention will be described. The inventor has determined that one way to improve control over the profile of the laser machined channels is to control the start-up of the motion of the feed table relative to turning on the laser beam. As is shown in FIG. 14, if the laser and table feed are initiated simultaneously, the lag in the motion of the table in the direction of arrow 180, which starts to move at time=t₀ relative to the illumination of the stent surface by the laser beam at time=t₁ results in a deeper portion, located between points A and B, being cut into the surface of the stent. Once the table starts to move at time=t₁, the depth of channel decreases and remains relatively constant until time=t_{f}, when the machining operation is completed for that channel.

While this inconsistency in channel depth in no way affects the utility of the channel to carry therapeutic substances, more consistent channels may be machined by simply introducing a delay into the programming instructions controlling the motion of the feed table and the laser beam. For example, by programming the table to start moving several milliseconds before turning on the laser beam, a more consistent channel depth can be achieved. In another embodiment, the laser and table are controlled to start simultaneously, by a n delay circuit controls the start up of the laser to delay the start of the laser a selected amount, thus providing an opportunity for the table to accelerate to a constant speed. For example, in one test carried out by the inventors, the laser was controlled to start up approximately 7.68 milliseconds after movement of the table was initiated.

A similar problem exists when the end of the channel is reached. As the table decelerates, the dwell time of the laser on the tubing becomes longer, resulting in more material being machined away at the end of the channel. In one embodiment of the present invention, the computer controlling the movement of the CNC positioning system is programmed to anticipate when the end of a channel is about to occur. At a predetermined point in time (or in location) before the end of the channel is reached, the computer turns off the laser, thus adjusting for the deceleration of the table, and providing for a more uniform machining of the tubing.

One example of machining channels and cutting a stent pattern will now be described. It will be understood that this description is merely exemplary, and it not intended to be limiting in any way. As shown in FIG. 4, a typical stent may include a number of rings, which may vary from two to as many as needed to provide a stent having a desired length. Starting at one end of the tubing, channels are machined into the portion of the tube which will eventually comprise the first two rings of the stent pattern. The positioning system then positions the laser relative to the tubing and the computer controls the position system and the laser to cut the pattern of the first ring. The positioning system is then controlled to position the tubing relative to the laser so that channels for the third ring in the sequence may be machined. The positioning system is then controlled to position the tubing relative to the laser so that the stent pattern of the second ring may be cut. This procedure is repeated until all of the channels and all of the rings have been machined and cut.

In another embodiment, the depth of the channels is controlled by passing the outer surface of the tubing under the laser one or more times. For example, depending on the depth of the channel desired, the tubing may be moved relative to the laser in a single pass, two passes, or three or more passes. As described above, the channels may be machined continuously, or the laser may be controlled to machine discrete channel portions. Thus, intermittent channels may be formed, or channels having varying depths may be formed. Additionally, the tubing may be passed under the laser beam in such a manner that portions of a channel are passed under the laser beam more than once. This provides the ability to selectively machine channels into a stent in a controlled manner, and thus also control the amount of drug that may be subsequently loaded into the channels and available for delivery by the stent.

It will be apparent from the foregoing that, while particular forms of the invention have been illustrated and described, various modifications can be made without departing from the spirit and scope of the invention. Accordingly, it is not intended that the invention be limited, except as by the appended claims.

1.-10. (canceled)

11. A system for forming a stent having one or more drug reservoirs incorporated therein, comprising:
a computer controlled movable positioning table;
a rotatable collet mounted on the positioning table, the collet configured to receive and hold a tubular member; the rotation of the collet also controlled by a computer;
a first a diode pumped Q-switched Nd/YAG laser assembly emitting a light beam for machining at least one channel into a portion of an outer surface of the tubular member; and
a second laser emitting a light beam capable of cutting a stent pattern into the tubular member so as to form a stent;
wherein the feed table is movable relative to the first and second laser light beams.

12. The system of claim 11, wherein the first laser is a diode pumped Q-switched Nd/YAG laser having a primary wavelength of approximately 1060 nanometers emitting a light beam having a wavelength equivalent to a third harmonic of the primary wavelength.

13. The system of claim 12, wherein the third harmonic wavelength is about 355 nanometers.

14. A system for forming a stent having one or more drug reservoirs incorporated therein, comprising:
a diode pumped Q-switched Nd/YAG laser assembly emitting a light beam having a wavelength equivalent to a third harmonic of a primary wavelength of the laser;
a computer controlled movable positioning feed table;
a rotatable collet mounted on the positioning table, the collet configured to receive and hold a tubular member; the rotation of the collet also controlled by a computer, the rotatable collet and movable positioning table operable to move in response to commands from the computer to move the tubular member relative to the light beam of the laser to machine at least one channel into a portion of an outer surface of the tubular member and to cut a stent pattern into the tubular member so as to form a stent, the channels being incorporated into at least one selected portion of the stent pattern.

15.-19. (canceled)
